(19) United States
(12) Patent Application Publication
(10) Pub. No.: US 2014/0094421 A1
(43) Pub. Date: Apr. 3, 2014

(54) HERBAL MEDICINAL COMPOSITION FOR TREATMENT OF WOMEN INFERTILITY AND A METHOD OF PREPARING THE SAME

(71) Applicant: FARIBA SEPAHVAND, Khoramabad (IR)

(72) Inventor: FARIBA SEPAHVAND, Khoramabad (IR)

(21) Appl. No.: 13/633,453

(22) Filed: Oct. 2, 2012

(51) Int. Cl. A61K 31/7034; A61P 15/08; A61P 31/04; A61P 31/12; A61P 31/10

(52) U.S. Cl. 514/25

(57) ABSTRACT

The embodiments herein provide a herbal medicinal composition for effectively treating a women infertility and a method for preparing the same. The composition comprises elm tree wood, a camel fat and water. The composition is prepared by taking several pieces of elm tree wood in a container and adding camel fat and water to the container. The container is heated for 120 minutes. The color of the contents of the container changes to bright red upon boiling. The mixture is filtered and cooled down at room temperature. The composition is taken by a patient in the morning for at least first seven days of the menstrual cycle.

### BACKGROUND

1. Technical Field

The embodiments herein generally relate to a treatment of uterus in women and particularly relate to a treatment of infertility in women. The embodiments herein more particularly relate to a herbal composition and a method of preparing the herbal composition for an effective treatment of women infertility.

2. Description of the Related Art

Ulmus glabra, the Wych elm or Scots elm, has the widest range of the European elm species ranging from Ireland eastwards to the Urals and from the Arctic Circle south to the mountains of the Peloponnese in Greece. The Wych elm trees are also found in Iran. The Wych elm trees are called by the name Maldj in Iran. The inner bark of the tree is astringent, demulcent and mildly diuretic. The inner bark is used both internally and externally in the treatment of diarrhoea, rheumatism, wounds, piles, etc. and is also used as a mouthwash in the treatment of ulcers. The inner bark is harvested from branches which are 3-4 years old and is dried for later use. A homeopathic remedy is also made from the inner bark of the Wych elm tree and is used for the treatment of eczema.

A camel is an even-toed ungulate within the genus Camelus bearing the distinctive fatty deposits known as humps on its back. The humps are actually a reservoir of fatty tissue. Concentrating the body fat in their humps minimizes heat-trapping and provides insulation throughout the rest of their body which may be an adaptation to living in hot climates. When this tissue is metabolized, it acts as a source of energy and yields more than 1 g of water for each 1 g of fat converted through a reaction with oxygen from air. This process of fat metabolization generates a net loss of water through respiration for the oxygen required to convert the fat.

The female infertility is defined as a failure to conceive in spite of having regular sexual intercourse for a year without using any contraceptive. The period of infertility varies with different people. The causes or factors of female infertility can be classified as whether they are acquired or genetic, or strictly by location. There are various factors which are responsible for infertility in women such as: endometriosis, polycystic ovary syndrome, uterine infections and pelvic inflammatory diseases. The age of women is also an important factor in determining an ability of women to conceive.

Many medical conditions can contribute to infertility. In fact, most cases of infertility are due to the underlying medical problems. These disorders can damage the fallopian tubes, interfere with ovulation or cause hormonal complications. A Pelvic inflammatory disease (PID) is a major cause of female infertility in worldwide. The PID comprises a variety of infections caused by different bacteria that affect the reproductive organs, appendix and parts of the intestine that lie in the pelvic area. The sites of infection most often implicated in infertility are in the fallopian tubes, a specific condition referred to as salpingitis. The PID may result from many different conditions that cause infections. Among them are: sexually transmitted diseases (cause of most PID), pelvic tuberculosis, nonsterile abortions and ruptured appendix. Chlamydia trachomatis is an infectious organism that causes 75% of infertility in the fallopian tubes. Gonorrhea is responsible for most of the remaining cases. Severe or frequent attacks of PID can eventually cause scarring, abscess formation and tubal damage that result in infertility. About 20% of women who develop symptomatic PID become infertile. The PID also significantly increases the risk of ectopic pregnancy (implantation of the embryo in the fallopian tubes). The severity of the infection, not the number of the infections, appears to pose the greatest risk for infertility.

Various methods have been developed to treat female infertility. These methods involve a use of drugs, conducting surgeries and other reproductive techniques. In-vitro Fertilization has been a boon to the medical sector with respect to the treatment of female infertility. But these methods are very expensive and result in generating many side-effects. Hence there is a need to come up with a cheap and an in-expensive method for a treatment of female infertility. Many herbal drugs have also been used for treating the women infertility but none of the prior art herbal drugs have used the properties of the Wych elm tree and the camel fat in combination to treat an infertility in women. Hence there is a need to develop a new herbal medicinal composition for a treatment of infertility in women.

The above mentioned shortcomings, disadvantages and problems are addressed herein and which will be understood by reading and studying the following specification.

### OBJECTIVES OF THE EMBODIMENTS

The primary object of the embodiments herein is to provide a composition for a treatment of infertility in women.

Another object of the embodiments herein is to provide a composition for a treatment of infertility in women suffering for long time with unknown reasons for many years.

Yet another object of the embodiments herein is to provide a composition that treats an infertility in women caused due to uterine infections, cysts, microbes and fungi.

Yet another object of the embodiments herein is to provide a composition to prepare the uterus of an infertile woman for fertility.

Yet another object of the embodiments herein is to provide an in-expensive composition that is simple, easy to prepare and does not have any side effects.

Yet another object of the embodiments herein is to provide a composition that uses the properties of elm tree wood along with a camel fat.

Yet another object of the embodiments herein is to provide a method of preparing a composition comprising elm tree wood and camel fat for a treatment of infertility in women.

These and other objects and advantages of the embodiments herein will become readily apparent from the following detailed description taken in conjunction with the accompanying drawings.

### SUMMARY

The various embodiments herein provide a herbal medicinal composition for a treatment of women infertility and a method of preparing a herbal medicinal composition for a treatment of infertility in women. The embodiments herein also provide a method of treating a woman suffering from infertility for many years.

According to one embodiment herein, an herbal medicinal composition for a treatment of women infertility comprises a plurality of pieces of an Elm tree wood, a camel fat, a plurality of additives and water. The plurality of pieces of the elm tree wood is younger branches of an Elm tree. An each piece of the Elm tree wood has a length of at least 5 cm. An each piece of the Elm tree wood weighs at least 50 grams. The plurality of pieces of the Elm tree wood is at least 10 in number. The plurality of pieces of Elm tree wood is kept in a lukewarm water at a room temperature for at least 2 days before use. An amount of the camel fat added to the composition is at least 250 grams. An amount of water required for every 10 pieces of the elm tree wood is with in a range of 2.5-3.5 liters, which is equal to 14 glasses of water. The plurality of additives includes tannin, resins, mucilages, phytosterols and stigmasterols. The tannin is a mixture of polyphenolic compounds. The tannin is a microbicide, a styptic, an anti-viral, an anti-fungal and removes infections from a uterus of a female. The composition is in a liquid form. The composition is bright red in color. The composition treats women suffering from chronic infertility.

According to an embodiment herein, a method of preparing a herbal medicinal composition for a treatment of female infertility comprises taking a plurality of pieces of an elm tree wood in a container. A predetermined amount of the camel fat is added to the container. A predetermined amount of water is added to the container to obtain a mixture. The mixture is heated for a predetermined time. The mixture is filtered and the camel fat and the plurality of pieces of the elm tree wood are separated to obtain a filtrate. The filtrate is cooled. The plurality of pieces of the elm tree wood is kept in a lukewarm water at a room temperature for at least 2 days before use. An each piece of the Elm tree wood has a length of at least 5 cm and has a weight of at least 50 grams. The predetermined amount of the camel fat added to the container is at least 250 grams. The predetermined amount of the water added to the container for every 10 pieces of Elm tree wood is an at least 2.5-3.5 litre which is equal to 14 glasses of water. The mixture is heated for a predetermined time of 120 minutes and preferably 100 minutes. The filtrate is kept in another container to cool down at a room temperature.

According to one embodiment herein, an herbal medicinal composition for a treatment of women infertility comprises a plurality of pieces of Elm tree wood, camel fat and water. The plurality of pieces of Elm tree wood is at least 10 in number. The plurality of pieces of Elm tree wood is kept in a lukewarm water at a room temperature for at least 2 days. An each piece of Elm tree wood is 5 cm in length. An each piece of Elm tree wood weighs 50 grams. The water added for every 10 pieces of elm tree is equal to at least 14 glasses. The amount of camel fat added to the composition is at least 250 grams. The composition is in a liquid form. The composition is bright red in color. The composition treats infertility in women suffering from many years.

According to an embodiment herein, a method of preparing a composition for the treatment of female infertility comprises taking a plurality of pieces of elm wood in a container. Further, a predetermined amount of camel fat is added to the container. A predetermined amount of water is added to the container to obtain a mixture. The mixture is then heated for a predetermined time. The mixture is filtered and cooled. The plurality of pieces of elm wood is kept in lukewarm water at a room temperature for at least 2 days before use. An each piece of Elm tree wood is of 5 cm in length. An each piece of Elm tree wood weighs 50 grams. The predetermined amount of camel fat added to the composition is at least 250 grams. The predetermined amount of water added for every 10 pieces of elm tree wood is at least 14 glasses. The mixture is heated for a predetermined time of 120 minutes. The predetermined time of heating is preferrably 100 minutes. The mixture is kept in another container to cool down at room temperature.

According to an embodiment herein, a method of treating infertility in women comprises administering a therapeutically effective amount of an herbal medicinal composition to a female patient. The herbal medicinal composition is administered every morning on the first seven days of a menstrual cycle. The therapeutically effective amount of the composition is at least 75 cc.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flow chart indicating the various steps involved in a method of preparing a herbal medicinal composition for a treatment of women infertility, according to an embodiment herein.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide an herbal medicinal composition for a treatment of women infertility and a method of preparing a herbal medicinal composition for a treatment of infertility in women. The embodiments herein also provide a method of treating a woman suffering from infertility for many years. The composition uses the therapeutic properties of a plant drug obtained from the woods of Wych elm tree in combination with camel hump fat.

According to one embodiment herein, the composition is obtained by heating a predetermined amount of Wych elm wood pieces with a predetermined amount of camel fat in water. The composition is a liquid composition. The composition is bright red or black cherry red in color. The composition treats uterine infections, uterus cysts and uterus microbic fungi and finally prepares the uterus for fertility.

According to an embodiment herein, an effective medicine is provided for the treatment of infertility or barrenness in women. The effective medicine is a mixture of camel fat and elm. The embodiments herein use elm and camel fat simultaneously to treat infertility.

According to the embodiments herein, the usable part of the Wych elm tree is the young branches of the tree. The young branches of the tree are cut from the centre in the form of small pieces. The pieces are kept in mild or lukewarm water at a room temperature. The pieces are kept in mild or lukewarm water at a room temperature so that the extracts come out easily while boiling.

The branches of Wych elm tree comprise many phyto-constituents. The Wych elm tree branches are mainly rich in tannins, resins, mucilage, phytosterol and stigmasterol. The most important and the most effective component for the treatment of infertility is the Tannin. Tannin is a mixture of polyphenolic compounds. Tannin is a microbicide, an anti-viral and an anti-fungal in nature. Tannin is also styptic and removes the infections from the uterus. The phyto-constituents of the Wych elm tree cause a putrefaction of the microbes, cysts and fungi present in the womb of an infertile women. These microbes, cysts and fungi come out of the woman's womb during the menstrual cycle.

The Camel fat is rich in stearic acid with 29% of palmitic acid, 27% of stearic acid and 26% of oleic acid as major components. The camel fat also contains myristic acid, arachidic acid, tetradecenoic acid, hexadecenoic acid, octadecadienoic acid and octadecatrienoic acid as minor components. Moreover, the camel fat contains some higher unsaturated fatty acids.

According to one embodiment herein, an herbal medicinal composition for a treatment of women infertility comprises a plurality of pieces of an Elm tree wood, a camel fat, a plurality of additives and water. The plurality of pieces of the elm tree wood is younger branches of an Elm tree. An each piece of the Elm tree wood has a length of at least 5 cm. An each piece of the Elm tree wood weighs at least 50 grams. The plurality of pieces of the Elm tree wood is at least 10 in number. The plurality of pieces of Elm tree wood is kept in a lukewarm water at a room temperature for at least 2 days before use. An amount of the camel fat added to the composition is at least 250 grams. An amount of water required for every 10 pieces of the elm tree wood is with in a range of 2.5-3.5 liters, which is equal to 14 glasses of water. The plurality of additives includes tannin, resins, mucilages, phytosterols and stigmasterols. The tannin is a mixture of polyphenolic compounds. The tannin is a microbicide, a styptic, an anti-viral, an anti-fungal and removes infections from a uterus of a female. The composition is in a liquid form. The composition is bright red in color. The composition treats women suffering from chronic infertility.

According to an embodiment herein, a method of preparing a herbal medicinal composition for a treatment of female infertility comprises taking a plurality of pieces of an elm tree wood in a container. A predetermined amount of the camel fat is added to the container. A predetermined amount of water is added to the container to obtain a mixture. The mixture is heated for a predetermined time. The mixture is filtered and the camel fat and the plurality of pieces of the elm tree wood are separated to obtain a filtrate. The filtrate is cooled. The plurality of pieces of the elm tree wood is kept in a lukewarm water at a room temperature for at least 2 days before use. An each piece of the Elm tree wood has a length of at least 5 cm and has a weight of at least 50 grams. The predetermined amount of the camel fat added to the container is at least 250 grams. The predetermined amount of the water added to the container for every 10 pieces of Elm tree wood is an at least 2.5-3.5 liter which is equal to 14 glasses of water. The mixture is heated for a predetermined time of 120 minutes and preferably 100 minutes. The filtrate is kept in another container to cool down at a room temperature.

According to one embodiment herein, an herbal medicinal composition for a treatment of women infertility comprises a plurality of pieces of Elm tree wood, camel fat and water. The plurality of pieces of Elm tree wood is at least 10 in number. The plurality of pieces of Elm tree wood is kept in a lukewarm water at a room temperature for at least 2 days. An each piece of Elm tree wood is 5 cm in length. An each piece of Elm tree wood weighs 50 grams. The water added for every 10 pieces of elm tree is equal to at least 14 glasses. The amount of camel fat added to the composition is at least 250 grams. The composition is in a liquid form. The composition is bright red in color. The composition treats infertility in women suffering from many years.

According to an embodiment herein, a method of preparing a composition for the treatment of female infertility comprises taking a plurality of pieces of elm wood in a container. Further, a predetermined amount of camel fat is added to the container. A predetermined amount of water is added to the container to obtain a mixture. The mixture is then heated for a predetermined time. The mixture is filtered and cooled. The plurality of pieces of elm wood is kept in lukewarm water at a room temperature for at least 2 days before use. An each piece of Elm tree wood is of 5 cm in length. An each piece of Elm tree wood weighs 50 grams. The predetermined amount of camel fat added to the composition is at least 250 grams. The predetermined amount of water added for every 10 pieces of elm tree wood is at least 14 glasses. The mixture is heated for a predetermined time of 120 minutes. The predetermined time of heating is preferrably 100 minutes. The mixture is kept in another container to cool down at room temperature.

According to an embodiment herein, a method of treating infertility in women comprises administering a therapeutically effective amount of an herbal medicinal composition to a female patient. The herbal medicinal composition is administered every morning on the first seven days of a menstrual cycle. The therapeutically effective amount of the composition is at least 75 cc.

According to one embodiment herein, a method of preparing an herbal composition comprises collecting a plurality of pieces of Wych elm tree branches. The plurality of pieces is collected by cutting the softer and younger branches of Wych elm tree into pieces. The inner part of the branch is more preferred. An each piece of Elm tree wood is cut into a size of 5 cm in length weighing at least 50 grams. The collected pieces of the Wych elm tree branch are kept in lukewarm water at room temperature for at least two days. Then, the pieces are taken into a dish. At least 10 pieces are preferably taken into the dish. Further, at least 250 grams of camel fat is added to the dish with simultaneous addition of at least 14 glasses of water. The dish is set on fire for heating. The contents of the dish are heated for at least 120 minutes on the flame. The contents of the dish are heated preferably for at least 100 minutes on the flame. When a temperature of the content reaches the boiling point, their color changes to bright red or black cherry. The contents are then filtered through a sieve or a muslin cloth to get a liquid composition. The liquid composition is then poured in another dish and kept at room temperature till the liquid cools down.

According to the embodiments herein, a method of preparing a herbal composition comprises:

Pieces of elm tree+camel fat+14 glasses of water→100 minutes of heating

According to an embodiment herein, a method of treatment of infertility in a woman comprises administering an effective amount of the liquid composition to the woman. The effective amount of the liquid composition is at least 75 cc. The liquid composition is taken early morning on the first seven days of the menstrual cycle.

The patient is advised to eat a teaspoon of composition in morning every day during the beginning period of 7 days in the menses time. Because this plant drug could perform putrefaction of womb especially cists of womb and womb, the microbes and the funguses go out of womb in the time of menses. So thus the womb is prepared for a pregnancy.

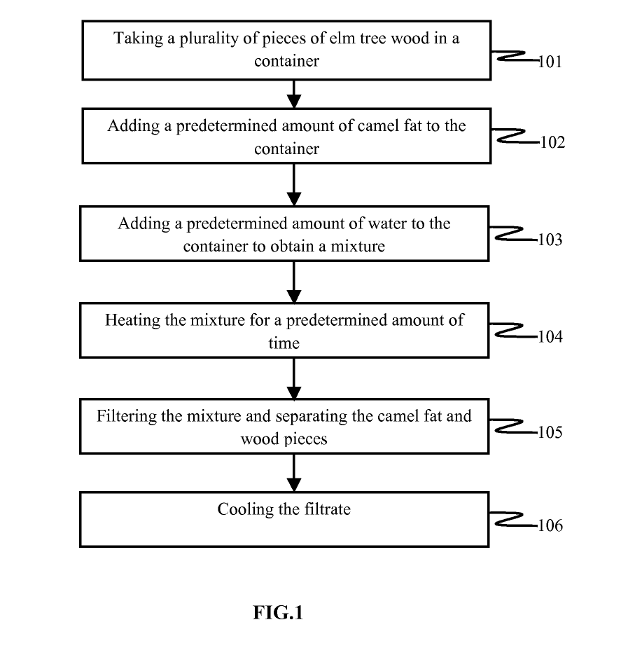

FIG. 1 illustrates a flow chart indicating the various steps involved in a method of preparation of an herbal medicinal composition for a treatment of women infertility, according to an embodiment herein. With respect to FIG. 1, a plurality of pieces of elm tree wood is taken in a container (101). The plurality of pieces of the elm tree wood comprises small pieces of the inner side of young branches of elm tree. The pieces are cut and soaked in water for at least 2 days at a room temperature before using. The pieces are cut into a size of 5 cm in length weighing 50 grams each. At least 10 pieces are preferably used. Then, a predetermined amount of camel fat is added to the container (102). The camel fat is obtained from the hump of a camel. The predetermined amount of the camel fat is at least 250 grams. A predetermined amount of water is added to the container to obtain a mixture (103). The predetermined amount of water added for every 10 pieces of elm tree wood is at least 14 glasses of water. The mixture is then heated for a predetermined time (104). The predetermined time of heating is at least 120 minutes. The predetermined time of heating is preferably at least 100 minutes. The color of the contents of the container turns into a bright red color upon boiling. The mixture is then filtered and separated from the camel fat and the wood pieces (105). The mixture is filtered through a sieve and the filtrate is obtained. The filtrate is cooled down at room temperature (106). The filtered mixture is kept in another dish or a container to cool down by its own at room temperature.

### Example 1

Every branch of the elm tree is cut into two pieces. The pieces are kept in lukewarm water at a room temperature for two days round the clock. The pieces are cut into a size of 5 cm in length weighing 50 grams each. For every 10 pieces of elm, at least 14 glasses of water is needed. According to the embodiments herein, the camel hump fat is used. The table 1 shows a composition of the herbal medicine effective for the treatment of infertility in woman according to the embodiments herein.

TABLE 1
Composition of the herbal medicine

| S. | | |
|---|---|---|
| No. | Ingredients | Amount |
| 1. | Elm wood | 10 pieces |
| 2. | Camel fat | 250 grams |
| 3. | Water | 14 glasses |

The ingredients according to table 1 are put in a container. The container is kept on heating. The heating is done for 120 minutes and preferably for 100 minutes. When boiling starts, the color of the ingredients turns into bright red. After heating, the mixture is filtered by passing it through a sieve to separate all the fat and wood pieces. The filtrate is kept in another container till it cools down.

### Direction for Usage

According to the embodiments herein, at least 75 cc of the composition should be taken by the patient every morning in the first seven days of a menstrual cycle. After taking the composition, the patient may feel the following symptoms: bleeding during the menstrual cycle, pain in breasts, feelings of breasts heaviness and toughness, pain in ovaries or pain in the pelvis. The composition treats uterine infections, uterus cysts and uterus microbic fungi and finally prepares the uterus for fertility.

### Experimental Data

The patients with long time infertility underwent some experiments regarding infertility. As there was no sample available, a case study was done. The sampling population included women who had sexual intercourse with no result.

It was decided to work on two volunteer women with 12 and 15 years of infertility to reach to scientific findings. Two women with 12 and 15 years of infertility who had used various infertility treatments with no result were treated with the herbal medicine according to the embodiment herein. The following side effects appeared in both patients: their menstrual cycle was intensified and uterine infections in the form of huge and heavy objects were seen. Their breasts became tough and heavy accompanied with pain in pelvis and ovary. After 25 days it was seen that the women were able to conceive.

Thus, the composition according to the embodiments herein is a herbal medicine that treats women infertility in the women suffering from many years. The composition kills the microbes present in the uterus and prepares the uterus for pregnancy. The composition is easy and simple to prepare and does not have any side effects. The composition is inexpensive and readily available.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A herbal medicinal composition for the treatment of women infertility comprises:
a plurality of pieces of an Elm tree wood;
a camel fat;
a plurality of additives; and
water.

2. The composition according to claim 1, wherein the plurality of pieces of the elm tree wood are younger branches of an Elm tree.

3. The composition according to claim 1, wherein the plurality of pieces of the Elm tree wood are at least 10 in number.

4. The composition according to claim 1, wherein the plurality of pieces of Elm tree wood are kept in a lukewarm water at a room temperature for at least 2 days before use.

5. The composition according to claim 1, wherein an each piece of the Elm tree wood has a length of at least 5 cm and wherein an each piece of the Elm tree wood has a weight of at least 50 grams.

6. The composition according to claim 1, wherein an amount of the camel fat is at least 250 grams.

7. The composition according to claim 1, wherein an amount of water required for every 10 pieces of the elm tree wood is within 2.5-3.5 litres.

8. The composition according to claim 1, wherein the plurality of additives are tannin, resins, mucilages, phytosterols and stigmasterols.

9. The composition according to claim 8, wherein the tannin is a mixture of polyphenolic compounds and wherein the tannin is a microbicide, a styptic, an anti-viral, an anti-fungal and removes infections from a uterus of a female.

10. The composition according to claim 1, wherein the composition is in a liquid form.

11. The composition according to claim 1, wherein the composition is bright red in colour.

12. The composition according to claim 1, wherein the composition treats women with chronic infertility.

13. A method of preparing a herbal medicinal composition for the treatment of female infertility comprises:
taking a plurality of pieces of an elm tree wood in a container;
adding a predetermined amount of a camel fat to the container;
adding a predetermined amount of water to the container to obtain a mixture;
heating the mixture for a predetermined time;
filtering the mixture and separating the camel fat and the plurality of pieces of the elm tree wood to obtain a filtrate; and
cooling the filtrate.

14. The method according to claim 13, wherein the plurality of pieces of the elm tree wood are kept in a lukewarm water at a room temperature for at least 2 days before use.

15. The method according to claim 13, wherein an each piece of the Elm tree wood has a length of at least 5 cm and wherein an each piece of the Elm tree wood has a weight of at least 50 grams.

16. The method according to claim 13, wherein the predetermined amount of the camel fat added to the container is at least 250 grams.

17. The method according to claim 13, wherein the predetermined amount of the water added to the container for every 10 pieces of the elm tree wood is within 2.5-3.5 litres.

18. The method according to claim 13, wherein the mixture is heated is for a predetermined time of 120 minutes and preferably 100 minutes.

19. The method according to claim 13, wherein the filtrate is kept in another container to cool down at a room temperature.

20. A method of treating infertility in females comprises: administering a therapeutically effective amount of an herbal medicinal composition to a female patient, wherein the herbal medicinal composition is administered every morning on a first seven days of a menstrual cycle, and wherein the therapeutically effective amount of the composition is at least 75 cc.
